(19) United States
(12) Patent Application Publication
(10) Pub. No.: US 2026/0110658 A1
(43) Pub. Date: Apr. 23, 2026

(54) METHODS AND APPARATUS FOR DETERMINING THE AMOUNT OF AN ANALYTE IN A FLUID

(71) Applicant: NUTROMICS TECHNOLOGY PTY LTD, Brunswick, Victoria (AU)

(72) Inventors: Alastair HODGES, Brunswick, Victoria (AU); Ali ALINEZHAD, Brunswick, Victoria (AU)

(21) Appl. No.: 19/118,981

(22) PCT Filed: Aug. 1, 2023
(86) PCT No.: PCT/AU2023/050717

§ 371 Date: Apr. 7, 2025

Related U.S. Application Data
(60) Provisional application No. 63/414,975, filed on Oct. 11, 2022.

(51) Int. Cl. G01N 27/327; G01N 27/416

(52) U.S. Cl. CPC G01N 27/3277; G01N 27/4168

(57) ABSTRACT

A method for determining an amount of an analyte in a fluid using an electrochemical aptamer-based sensor. The method includes applying a step-wise change in potential to a working electrode of the sensor, and determining a distribution of redox reporters of the aptamers from the current values measured. The amount of analyte about the sensor is then determined from the distribution.

### FIELD OF THE INVENTION

The present invention relates generally to electrochemical sensors useful in determining the amount of an analyte in a biological fluid such as blood and interstitial fluid. More particularly, the invention provides for an electrochemical sensor that is operated by way of an improved chronoamperometry method which provides for higher reliability analyte determination across a range of conditions.

### BACKGROUND TO THE INVENTION

Some classes of electrochemical sensors are selective and capable of the real-time, continuous detection of target analytes, as well as single point measurements, including agents which are exogenous (e.g., pharmaceutical compounds and toxins) and also those which are endogenous (e.g., metabolites, proteins, hormones, and the like).

These sensors may comprise an electrode with an associated binding element that undergoes a conformational change upon analyte binding. The conformational change alters the accessibility of a redox reporter to the electrode surface, thereby producing an analyte-induced change in the electron exchange between the redox reporter and the electrode. In some circumstances, binding of the analyte brings the redox reporter proximal to the electrode surface, thereby increasing the speed of electron exchange and in turn increasing current through the electrode. In other circumstances, binding moves the reporter distal to the electrode surface resulting in the opposite effects. Regardless, binding of the analyte results in a detectable change in electrode current.

Electrochemical sensors may be embodied in many forms, one of which is a microneedle-based patch applied to skin such that the electrode contacts the interstitial fluid. The tip of the microneedle functions as the sensor electrode, with the binding element being associated with the tip. This arrangement provides a minimally invasive platform for real-time, continuous in vivo target analyte detection, which is sufficiently sensitive and selective to function in the complex matrix of the interstitial fluid.

Whilst prior art sensors are undoubtedly useful, they present a number of problems.

For example, the response of whole blood glucose sensors can alter according to the viscosity of the blood, which in turn can change according to temperature, haematocrit, and lipid content. In most devices in the prior art such undesired variation is addressed by using correction factors when calculating analyte amount. A separate temperature sensor may be provided, the sensor being calibrated having regard to a pool of population data.

Signal drift is a significant problem in continuous electrochemical sensors. Prior artisans have proposed methods for quantitating bound analyte using electrochemical square wave voltammetry, whereby measurements and multiple square wave frequencies are used to account for signal drift over time. However, such methods require the careful choice of particular frequency pairs where the drift of signal at the two frequencies is empirically matched. There remains uncertainty whether or not the chosen frequencies are applicable to all situations, for example at different temperatures or for different devices subject to manufacturing variation.

Further problems arise from variations in sensor output arising from electrode area, layer thickness and redox species diffusion coefficient and density adjacent to the electrode surface.

This invention seeks to overcome, or at least ameliorate, these deficiencies in the prior art by providing an easy to implement and extremely fast method whereby quantitation of the amount of analyte bound proximal or distal to an electrode can be determined in a way that is more immune to variations in the sensing environment and sensor to sensor manufacturing variations, as well as changes to the device over time, both before and during use.

It is an aspect of the present invention to provide an improvement to the operation of electrochemical sensors so as to ameliorate or overcome any one or more of undesirable variations in output, inaccurate output, imprecise output, and unreproducible output. It is a further aspect of the present invention to dispense with ancillary means in electrochemical sensors for addressing any one or more of undesirable variations in output, inaccurate output, imprecise output, and unreproducible output. It is a further aspect of the present invention to provide a useful alternative to prior art methods of interrogating electrochemical sensors.

The discussion of documents, acts, materials, devices, articles, and the like, is included in this specification solely for the purpose of providing a context for the present invention. It is not suggested or represented that any or all of these matters formed part of the prior art base or were common general knowledge in the field relevant to the present invention as it existed before the priority date of each provisional claim of this application.

### SUMMARY OF THE INVENTION

In a first aspect, but not necessarily the broadest aspect, the present invention provides a method for determining an amount of an analyte in a fluid, the method comprising:
applying a first change in potential to an electrochemical sensor working electrode, the electrode having associated therewith (i) a binding element and (ii) an associated redox-active species spatially constrained within a layer adjacent to the electrode surface;
measuring current values resulting from at least the application of the first change in potential at a plurality of time points;
determining a distribution of the redox-active species by reference to the measured current values; and
determining the amount of an analyte bound to the binding element using the determined distribution of the redox-active species.

In one embodiment of the first aspect, the change in potential is substantially instantaneous.

In one embodiment of the first aspect, the change in potential is effected as a step-wise change.

In one embodiment of the first aspect, first change in potential is configured to change the redox state of the redox-active species from one redox state to another redox state such that electrons are transferred between the redox-active species and the electrode surface.

In one embodiment of the first aspect, the first change in potential is configured such that the current flowing through the electrode surface as a result of the redox-active species changing redox state is controlled by mass transport of the redox-active species through the layer adjacent to the electrode surface.

In one embodiment of the first aspect, the potential resulting from the first change in potential is maintained for at least about 10 ms, 20 ms, 30 ms, 40 ms, 50 ms, 60 ms, 70 ms, 80 ms, 90 ms, or 100 ms.

In one embodiment of the first aspect, the method comprises applying an initial potential prior to the application of the first change in potential.

In one embodiment of the first aspect, the initial potential is selected such that the magnitude of the first change in potential is at least about 0.1 V, 0.2 V, 0.3 V, 0.4 V, 0.5 V, 0.6 V, 0.7 V, 0.8 V, 0.9 V, or 1.0 V.

In one embodiment of the first aspect, the initial potential is maintained for at least about 0.05 s, 0.1 s, 0.2 s, 0.3 s, 0.4 s, 0.5 s, 0.6 s, 0.7 s, 0.8 s, 0.9 s, 1.0 s, 1.1 s, 1.2 s, 1.3 s, 1.4 s. 1.5 s, 1.6 s, 1.7 s, 1.8 s, 1.9 s, or 2.0 s.

In one embodiment of the first aspect, the method comprises measuring at least a first current value subsequent to the application of the first change in potential.

In one embodiment of the first aspect, the first current value is measured substantially immediately subsequent to the application of the first change in potential.

In one embodiment of the first aspect, the first of the first current value is measured less than about 0.1 ms, 0.2 ms, 0.3 ms, 0.4 ms, 0.5 ms, 0.6 ms, 0.7 ms, 0.8 ms, 0.9 ms, or 1.0 ms, subsequent to the application of the first change in potential.

In one embodiment of the first aspect, the method comprises measuring at least a first and a second current value in chronological order subsequent to the application of the first change in potential.

In one embodiment of the first aspect, the method comprises measuring at least a first, a second and a third current value in chronological order subsequent to the application of the first change in potential.

In one embodiment of the first aspect, the method comprises measuring at least a first, a second, a third, and a fourth value current value in chronological order subsequent to the application of the first change in potential.

In one embodiment of the first aspect, measurement of the first current value, the second current value (where measured), the third current value (where measured) and the fourth current value (where measured) occurs within the period that the potential resulting from the first change in potential is maintained.

In one embodiment of the first aspect, the first current value, the second current value (where performed), the third current value (where measured) and the fourth current value (where measured) is an averaged current value, the averaged current value determined by reference to a plurality of current values determined up to, across, or after a time point.

In one embodiment of the first aspect, the average current value is determined by reference to at least 2, 3, 4, 5, 6, 7, 8, 9, 10, 15, 20, 25, or 30 current values.

In one embodiment of the first aspect, the method comprises generating at least one current ratio by reference to a first current value and a second current value.

In one embodiment of the first aspect, the first current value is measured substantially immediately after application of the first change in potential.

In one embodiment of the first aspect, the first current value is measured within about 0.1 ms, 0.2 ms, 0.3 ms, 0.5 ms, 0.5 ms, 0.6 ms, 0.7 ms, 0.8 ms, 0.9 ms, 1.0 ms, 2.0 ms, 3.0 ms, 4.0 ms, or 5.0 ms, after application of the first change in potential.

In one embodiment of the first aspect, the second current value is measured after the first current value.

In one embodiment of the first aspect, the second current value is measured within about 1.0 ms, 2.0 ms, 3.0 ms, 4.0 ms, 5.0 ms, 6.0 ms, 7 ms, 8 ms, 9 ms, 10 ms, 11 ms, 12 ms, 13 ms, 14 ms, 15 ms, 16 ms, 17 ms, 18 ms, 19 ms, 20 ms, 21 ms, 22 ms, 23 ms, 24 ms, or 25 ms, after the first current value is measured, or after application of the first change in potential.

In one embodiment of the first aspect, the method comprises applying a second change in potential to the electrochemical sensor working electrode after the first change in potential.

In one embodiment of the first aspect, the sign of the second change in potential is the same as the sign of the first change in potential.

In one embodiment of the first aspect, the sign of the second change in potential is opposite to the sign of the first change in potential.

In one embodiment of the first aspect, the second change in potential is used to estimate the capacitive charging current and current due to redox species not confined to the layer adjacent to the electrode, and therefore background current draw, of the sensor apparatus.

In one embodiment of the first aspect, the background current draw is subtracted from the first current value, the second current value (where measured), the third current value (where measured) or the fourth current value (where measured). Optionally, the background current draw may be multiplied by a factor prior to subtracting it from the first, second, third or fourth current value, where they are measured.

In one embodiment of the first aspect, the first and/or second change in potential is a substantially instantaneous change.

In one embodiment of the first aspect, the first and/or second change in potential is substantially a step-wise change.

In one embodiment of the first aspect, the method comprises the steps of:
applying a first change in potential and at least a second change in potential;
combining selected currents resulting from the at least second change in potential with selected currents from the first change in potential to derive a current; and
determining an analyte concentration from the derived current.

In one embodiment of the first aspect, the at least second change in potential changes from the potential applied at the end of the first change in potential.

In one embodiment of the first aspect, the selected currents are combined by subtracting a function of the magnitude of the current resulting from the at least second change in potential from the magnitude of the current resulting from the first change in potential, where the time the current was sampled after the change in potential is substantially the same for both the first and the at least second change in potential.

In one embodiment of the first aspect, the magnitude of the current resulting from the at least second change in potential is multiplied by a factor and subtracted from the current resulting from the first change in potential.

In one embodiment of the first aspect, the method comprises the step of combining the currents resulting from the first change in potential and the at least second change in potential by using the currents measured at the same time after the change in potential and the size of the change in potential used to generate the currents, to extrapolate the measured currents to a value corresponding to a zero change in potential.

In one embodiment of the first aspect, the extrapolation is a linear extrapolation.

In one embodiment of the first aspect, the extrapolation is a non-linear extrapolation.

In a second aspect, the present invention provides an electrochemical sensor apparatus or system comprising:
a working electrode having associated therewith (i) a binding element and (ii) an associated redox-active species spatially constrained within a layer adjacent to the electrode surface; and
a microprocessor-based controller,
wherein the microprocessor-based controller is configured to perform the method of any embodiment of the first aspect.

In one embodiment of the second aspect, the microprocessor-based controller is in electrical connection with the working electrode, or in wired or wireless network connection with another microprocessor-based controller that is in electrical connection with the working electrode.

In one embodiment of the second aspect, the microprocessor controller is configured to access and execute program instructions to perform the method of any embodiment of the first aspect.

In one embodiment of the second aspect, the electrochemical sensor apparatus or system comprises a variable power supply in electrical connection with the working electrode, wherein the program instructions direct the power supply to apply a potential to the working electrode according to the method of any embodiment of the first aspect.

In one embodiment of the second aspect, the electrochemical sensor apparatus or system comprises current measuring circuitry configured to measure an electrical current through the working electrode.

In one embodiment of the second aspect, the electrochemical sensor apparatus or system comprises electronic memory in operable association with the microprocessor-based controller and the current measuring circuitry, the electronic memory configured to store one or more currents measured by the current measuring circuitry.

In one embodiment of the second aspect, the microprocessor-based controller is configured to process the one or more currents measured by the current measuring circuitry to provide an analyte amount.

In a third aspect, the present invention provides computer-readable medium comprising program instructions configured to execute the method of any embodiment of the first aspect.

### BRIEF DESCRIPTION OF THE FIGURES

FIG. 1 is a graph of f value versus the concentration of vancomycin (in micromolar units). The points are the calculated f values, corrected for the f value when no vancomycin is present. The curved line shown is given by fitting the Langmuir binding isotherm equation to the data. This graph represents the response of a gold electrode coated with vancomycin aptamer and was tested at 32° C.

FIG. 2 is a graph of f value versus the concentration of vancomycin (in micromolar units). The points are the calculated f values, corrected for the f value when no vancomycin is present. The curved line shown is given by fitting the Langmuir binding isotherm equation to the data. The graph represents the response of a gold electrode coated with vancomycin aptamer and was tested at 41° C.

FIG. 3 is a graphical representation of the potentials applied in the course of experiments detailed in Example 1.

FIG. 4 is a graph of f value versus time (in minutes). The graph shows f values and current values taken at t₁ (0.5 ms) for different electrodes interrogated under different temperature conditions. f values were corrected for background response.

FIG. 5 is a graph of current (in microamps) versus time. The graph shows the current over time for a sensor where five different potential step sizes are used.

FIG. 6 is a graph of current (in microamps) versus potential step size (in volts). The graph shows examples of the sensor current at particular times plotted against the size of the potential step used.

FIG. 7 is a graph of current (in microamps) versus time (in seconds). The graph shows the current over time for one of the potential step sizes for various vancomycin concentrations.

FIG. 8 is a graph of current (in microamps) versus time (in seconds). The graph shows the current extrapolated to zero potential step size over time for various vancomycin concentrations.

FIG. 9 is a graph of f value versus the concentration of vancomycin (in micromolar units). The graph shows the f values calculated using the current from one of the potential step sizes and from the current extrapolated to zero potential step size, for various vancomycin concentrations. f values were corrected for background response.

FIG. 10 is a graph of current (in microamps) versus time (in seconds). The graph shows the current over time for a first change in potential for various vancomycin concentrations.

FIG. 11 is a graph of current (in microamps) versus time (in seconds). The graph shows the current over time for a second change in potential, starting at the end potential of the first change in potential that used to generate the currents shown in FIG. 10, for various vancomycin concentrations.

FIG. 12 is a graph of current (in microamps) versus time (in seconds). The graph shows the current over time where the current from the second change in potential has been multiplied by 2.5 and subtracted from the current from the first change in potential.

FIG. 13 is a graph off value versus the concentration of vancomycin (in micromolar units). The graph shows the f values calculated using the currents from FIG. 10 and using the currents from FIG. 12, for various vancomycin concentrations. f values were corrected for background response.

Unless otherwise indicated herein, features of the drawings labelled with the same numeral are taken to be the same features, or at least functionally similar features, when used across different drawings.

With the exception of the graphs, the drawings are not prepared to any particular scale or dimension and are not presented as being a completely accurate presentation of the various embodiments.

### DETAILED DESCRIPTION OF THE INVENTION AND PREFERRED EMBODIMENTS THEREOF

After considering this description it will be apparent to one skilled in the art how the invention is implemented in various alternative embodiments and alternative applications. However, although various embodiments of the present invention will be described herein, it is understood that these embodiments are presented by way of example only, and not limitation. As such, this description of various alternative embodiments should not be construed to limit the scope or breadth of the present invention. Furthermore, statements of advantages or other aspects apply to specific exemplary embodiments, and not necessarily to all embodiments, or indeed any embodiment covered by the claims.

Throughout the description and the claims of this specification the word “comprise” and variations of the word, such as “comprising” and “comprises” is not intended to exclude other additives, components, integers, or steps.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may.

Where the term “determine”, “determining” and “determined” are used, it is not intended that these terms be necessarily interpreted to mean any completely accurate determination, although that meaning is not excluded. The terms may be interpreted to include an estimation, an approximation or even an indication.

In a first aspect, the present invention provides a method for determining an amount of an analyte in a fluid, the method comprising:
applying a first change in potential to an electrochemical sensor working electrode, the electrode having associated therewith (i) a binding element and (ii) an associated redox-active species spatially constrained within a layer adjacent to the electrode surface;
measuring current values resulting from at least the application of the first change in potential at a plurality of time points;
determining a distribution of the redox-active species by reference to the measured current values; and
determining the amount of an analyte bound to the binding element using the determined distribution of the redox-active species.

In some embodiments of the method, the potential applied to the working electrode of the apparatus is changed from one that is sufficient to convert and maintain the redox species in one redox state, to another that is sufficient to substantially immediately change the redox state of any redox-active species sufficiently close to the electrode to allow electron transfer across the electrode/solution interface. Expressed in an alternative manner, the potential is changed to one such that the current flowing through the electrode surface as a result of the redox-active species changing redox state is controlled predominantly or substantially by mass transport of the redox-active species toward the electrode. As a result of the change in potential, net electrical current flows through the electrode surface, typically with the current changing in magnitude over time.

Without wishing to be limited by theory in any way, it is proposed that a redox-active reporter that is tethered to the working electrode surface (by an aptamer, for example), and therefore capable of restricted movement only within a layer adjacent the electrode, the redox-active species nevertheless substantially freely diffuses within the layer. Accordingly, movement of the redox-active species may be modelled with some accuracy (although not necessarily complete accuracy) on Fick's first and second laws of diffusion. Thus, even though tethered the redox-active species may nevertheless move from a region of high concentration to a region of low concentration, the magnitude of the flux being proportional to the differences in concentration, in a manner expected for a freely diffusible (i.e., untethered) species. Moreover, the concentration gradient of the redox-active species in the layer changes over time in a manner expected for a freely diffusible species.

Fick's first and second laws were applied to a system whereby a redox-active species is spatially constrained to be within a layer adjacent to the electrode surface, but able to freely diffuse within that layer. Accordingly, it was possible to model diffusion of the constrained redox species, and therefore model changes in current over time (termed “current transient”) due to movement of the redox-active species relative to the electrode surface given that electron transfer increases as the redox-active species is more proximal to the surface.

The resulting modelled current transient resulting from movement of the redox-active species revealed a dependence on (i) the surface area of the electrode, (ii) the thickness of the layer adjacent to the electrode, (iii) the diffusion coefficient of the redox species in the layer adjacent to the electrode, (iv) the overall concentration of the redox species in layer adjacent to the electrode and (v) the distribution of the redox species within the layer adjacent to the electrode at the time the potential step was applied. These revelations have been found to be of practical consequence in relation to methods for the interrogation of an electrochemical sensor apparatus, the determination of redox-active species distribution, and in turn determination of the amount of an analyte bound to the binding element.

Further investigations were carried out demonstrating the practical relevance of single current measurements and current ratios determined in the period immediately after application of the potential change. It was surprisingly found that at longer times after the change in potential was applied, a ratio of two measured currents was strongly dependent upon the thickness of the layer adjacent to the electrode and the diffusion coefficient of the redox species but only weakly dependent upon the initial distribution of the redox species, whereas the ratio of a current at a shorter time after the potential step was applied, compared to a current at a longer time, was more strongly dependent upon the initial distribution of the redox species as well strongly dependent upon the thickness of the layer adjacent to the electrode and the redox species diffusion coefficient. In addition, both these current ratios were modelled to be insensitive to the overall concentration of the redox species and the electrode area.

These modelled behaviours suggested that the later current ratio may be useful in obtaining a combined measure of the redox species diffusion coefficient in combination with the thickness of the layer adjacent to the electrode, which could be applied to the earlier current ratio to obtain an estimated measure of the initial distribution of the redox species, where the estimate is largely insensitive to changes in the overall concentration of the redox species in the layer adjacent to the electrode, the thickness of the layer adjacent to the electrode and the diffusion coefficient of the redox species in the layer adjacent to the electrode and the area of the electrode.

The equation for current over time derived from the model is shown as Equation (1), below:

$$i ( t ) = \frac{4 zFAD {C}_{0}}{l} {\sum }_{n = 0}^{∞} ( f ( 1 - \frac{4 {( - 1 )}^{n}}{( 2 n + 1 ) π} ) + \frac{2 {( - 1 )}^{n}}{( 2 n + 1 ) π} ) {e}^{- {( 2 n + 1 )}^{2} {π}^{2} Dt / 4 {l}^{2}}$$

where:
i(t) is the electrical current at time t,
z is the number of moles electrons transferred per mole of redox species either oxidised or reduced at the electrode surface,
F is Faraday's Constant,
A is the area of the electrode,
D is the diffusion coefficient of the redox species in the layer adjacent to the electrode,
C₀ is the overall concentration of the redox species in the layer adjacent to the electrode,
l is the thickness of the layer containing the redox species adjacent to the electrode, and
f is the fraction of the redox species that is close to or at the electrode surface when the potential step is applied.

Thus, f may be used to determine the distribution of the redox-active species when an initial potential is applied, and in the instant before the change in potential is applied. The distribution of the redox-active species may be used to determine the amount of analyte bound to the binding element of the sensor.

Note from Equation (1) that by taking the ratio of currents at two different times the electrode area and redox species concentration terms cancel out. In addition, at a sufficiently long time the exponential terms with n>0 will be small enough relative to the n=0 exponential term such that Equation (1) can be approximated by Equation (2), as follows:

$$i ( t ) \sim \frac{4 zFAD {C}_{0}}{l} ( f ( 1 - \frac{4}{π} ) + \frac{2}{π} ) {e}^{- {π}^{2} Dt / 4 {l}^{2}} \cdot for \cdot t \cdot > \cdot {t}_{min} \qquad ( 2 )$$

Accordingly, by taking the ratio of current at two different times where Equation (2) applies, only the exponential term does not cancel out, so that an estimate of D/l² may be obtained.

According to this method, the current at least three different times during the current transient may be determined: i(t₁), i(t₂) and i(t₃). Optionally, a current at a fourth time i(t₄) may be determined. Time t₁ is selected to be a short time after the change in potential step is applied, t₃ and optionally t₄ at longer times after the potential step is applied and t₂ to be between t₁ and t₃ or optionally to be between t₁ and t₄.

At least two ratios of currents may then be determined. In a preferred embodiment of the invention the ratios i(t₁)/i(t₃) and i(t₂)/i(t₃) are determined. In other embodiments of the invention, the ratios i(t₁)/i(t₄) and/or i(t₂)/i(t₄) are determined. In some embodiments of the invention, current ratios at additional times can be calculated and used to improve the method by providing additional estimates of the derived parameters. The optional ratios i(t₁)/i(t₄) and/or i(t₂)/i(t₄) can be used to calculate additional estimates of/values, to assist in the verification or accuracy of that calculated using the i(t₁)/i(t₃) and i(t₂)/i(t₃) ratios.

In a further embodiment of the method, a second change in electrode potential is performed, after the first change in electrode potential. In this embodiment, the electrode is held at the potential resulting from the first change in potential and for a sufficient time to substantially electrochemically oxidise or reduce all the redox-active species present in the tethered layer. The electrode potential is then changed in the direction of the initial potential. For example, if the electrode potential was initially held at a value where the redox-active species was reduced, the second potential change would be in the direction of a stronger reducing potential. If the electrode potential was initially held at a value where the redox-active species was oxidised, the second step would be in the direction of a stronger oxidising potential. The potential applied after the second step is chosen such that the faradaic current arising from the sensing redox species is substantially absent from the current flowing after this potential is applied. The current resulting from the second potential change can be used to obtain an estimate of the non-faradaic current flowing at the electrode, due to capacitive double-layer charging for example as well as any faradaic current from redox species that are not confined in the layer adjacent to the electrode, and subtracted from the current used to calculate the current ratios disclosed above, to improve the accuracy of the results. The current from the second potential change can optionally be multiplied by a factor before it is subtracted from the current used to calculate the current ratios disclosed above. In an alternative to this embodiment, the second change in the electrode potential is in the direction opposite to the direction of the initial potential. The potential applied after the second step is chosen such that the faradaic current arising from the sensing redox species is substantially absent from the current flowing after this potential is applied. To achieve this according to this alternative, the potential step should be chosen such that it does not substantially change the redox state of the sensing redox species. In this alternative the current flowing after the second potential step is applied will be opposite in sign to that flowing after the first potential step is applied. The current from the second potential change can optionally be multiplied by a factor before it is added to the current used to calculate the current ratios disclosed above, to improve the accuracy of the results. This alternative can be desirable in situations where, for example, stepping to a stronger oxidizing potential could result in additional faradaic current flowing that is not related to the sensing redox species.

In a further embodiment of the invention, multiple steps in potential of different magnitude are applied to the sensor. According to this embodiment at least two potential steps are applied, where the starting potential or the ending potential of the potential step is different for the different potential steps. Merely for illustration purposes, if the starting potential for a first step was −0.4 V and the ending potential −0.15 V, the second potential step could start at −0.45 V and end at −0.15 V and a third potential step, if used, start at −0.5 V and end at −0.15 V. Alternatively, the first step could start at −0.4 V and end at −0.15 V, the second potential step start at −0.4 V and end at −0.1 V and the third potential step, if used, start at −0.4 V and end at −0.05 V. The difference in the starting potential or ending potential between the multiple potential steps is chosen so that the current contributed by faradaic electron transfer across the electrode interface contributed by the sensing redox species does not substantially vary for the different potential steps, but where the capacitive charging current and possible extraneous faradaic current varies in proportion to the potential step size. A first current value from the first potential step at a chosen time t₁ is combined with first current value at time t₁ from the second and any subsequent potential steps, to extrapolate the first current value to a theoretical value at a zero potential step size. Any appropriate extrapolation function can be used, where the technique used is appropriate for the type of dependence of the current versus potential step size. For example, if the current measured varies linearly with potential step size, linear least squares regression can be applied to the current values at the chosen time for the various potential steps versus the size of the potential step and the regression intercept used as the extrapolated current at zero potential step size. The extrapolated current at zero potential step size is used as an estimate of the sensing redox species faradaic current at this time, substantially free of interference with currents that vary in magnitude with potential step size, such as capacitive charging currents and faradaic currents that are electron transfer rate controlled. When more than two different potential step sizes are used in this embodiment, the degree of correlation in the extrapolation plot can be used as a measure of correct functioning of the device. The extrapolation process is repeated for the current values at the second and third times and at a fourth time where measured and the extrapolated values used to calculate f values as given elsewhere in this disclosure.

An advantage of some embodiments of the invention is that a method is provided which allows the distribution of the redox species to be estimated without specific knowledge of the electrode area, thickness of the tethered redox layer, overall amount of redox species, or number of electrons transferred per mole of redox species. A chronoamperometry method that can achieve this has not previously been disclosed to the best of Applicant's knowledge. Moreover, a method using simple ratios of currents at different times to derive the distribution was hitherto unknown.

Another advantage of some embodiments of the invention is that a chronoamperometry method is provided which is significantly faster than prior art interrogation methods (such as square wave voltammetry methods), which may take seconds to minutes to execute. The presently described methods, in some embodiments, can be executed in milliseconds to tens of milliseconds. For example, the electrode is optionally held at an initial potential for a short time (of the order of milliseconds to a second), and then the potential changed in a step-wise manner and held at a second potential for a time, typically up to tens of milliseconds. Because of the speed of execution and the fact that the redox species is tethered to the electrode, the stepping of potential can be repeated multiple times over a short period to obtain multiple estimates of the desired parameters that can be averaged or otherwise combined to reduce random variation in the results.

In one embodiment of the invention, multiple steps in potential of different magnitude are applied to the sensor. According to this embodiment, at least two potential steps may be applied, where the starting potential or the ending potential of the potential step is different for the different potential steps. The first current value from the first potential step may be combined with first current value from the second and any subsequent potential steps to extrapolate to an extrapolated value at a zero potential step size. This process may also be repeated for the second, third and fourth current values where measured.

The difference in the starting potential or ending potential between the multiple potential steps may be chosen so that the current contributed by oxidizing or reducing the sensing redox species does not substantially vary for the different potential steps, but where the capacitive charging current and other possible extraneous currents varies with the potential step size.

If the capacitive charging current and other possible extraneous currents are proportional to the potential step size, a linear extrapolation method such as linear least squares can be used to regress the measured current against the potential step size, with the intercept of this regression used as the extrapolated value.

If the capacitive charging current and/or other possible extraneous currents vary in a non-linear fashion with the potential step size, a non-linear extrapolation method such as is known in the art may be used to regress the measured current against the potential step size, with the intercept of this regression used as the extrapolated value. Extrapolating the measured current versus potential step size to zero potential step size may remove or substantially reduce the contribution of the capacitive charging current and other extraneous currents that vary with potential step size to the extrapolated current values obtained. This in turn may improve the ability of the extrapolated current to discriminate between different target analyte concentrations, as a greater fraction of the extrapolated current is due to the presence of the target analyte, compared to the circumstance where non-extrapolated current is used as a measure of the target analyte concentration.

The present invention may be embodied in the form of an apparatus or a system, configured to facilitate execution of the methods described herein.

An apparatus of the present invention may be embodied in the form of a wearable device that is substantially self-contained, allowing measurements to be performed whilst the subject is undergoing normal activities and/or over a prolonged period of time. The wearable device may be a collar, a bracelet, a strap, an adhesive, or a patch. The wearable device may include transdermal microneedles, one of which functions as the working electrode of the sensor by contacting the interstitial fluid of the subject and detecting analytes therein.

The wearable device may further comprise a housing structure enclosing one or more other components, such as a processor-based microcontroller. The controller is configured to be in electrical communication with at least one electrode, and generally would include a power source, a data processing unit, electronic memory, and a wireless transmitter/receiver.

When embodied as a system, components may be distributed in different physical locations although still operate in an integrated manner. For example, software instructions may be stored and executed by a smart phone or other remote process in data communication with the microprocessor-based controller in a wearable device.

As will be understood, the methods described herein may be deployed in part or in whole through one or more microprocessors that execute computer software, program codes, and/or instructions on a processor. A microprocessor may be any kind of computational or processing device capable of executing program instructions, codes, binary instructions, and the like.

Any microprocessor may access a storage medium (such as electronic memory) through an interface that may store methods, codes, and instructions as described herein and elsewhere. The storage medium associated with the processor for storing methods, programs, codes, program instructions or other type of instructions capable of being executed.

The computer software, program codes, and/or instructions may be stored and/or accessed on computer readable media that may include: computer components, devices, and recording media that retain digital data used for computing for some interval of time; semiconductor storage known as random access memory (RAM); mass storage typically for more permanent storage, such as non-volatile memory such as read only memory (ROM).

The methods described herein may transform physical and/or or intangible items from one state to another. The methods and systems described herein may also transform data representing physical and/or intangible items from one state to another.

Software products may be created using a structured programming language such as C, an object oriented programming language such as C++, or any other high-level or low-level programming language (including assembly languages, hardware description languages, and database programming languages and technologies) that may be stored, compiled or interpreted to run on a microprocessor, as well as heterogeneous combinations of processors, processor architectures, or combinations of different hardware and software, or any other machine capable of executing program instructions.

Thus, in one aspect, any method may be embodied in computer executable code that, when executing on one or more microprocessors, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

The invention may be embodied in program instruction set executable on one or more microprocessors. Such instructions set may include any one or more of the following instruction types.

Data handling and memory operations, which may include an instruction to set a register to a fixed constant value, or copy data from a memory location to a register, or vice-versa, to store the contents of a register, result of a computation, or to retrieve stored data to perform a computation on it later, or to read and write data from hardware devices.

Arithmetic and logic operations, which may include an instruction to add, subtract, multiply, or divide the values of two registers, placing the result in a register, possibly setting one or more condition codes in a status register, to perform bitwise operations, e.g., taking the conjunction and disjunction of corresponding bits in a pair of registers, taking the negation of each bit in a register, or to compare two values in registers (for example, to determine if one is less, or if they are equal).

Control flow operations, which may include an instruction to branch to another location in the program and execute instructions there, conditionally branch to another location if a certain condition holds, indirectly branch to another location, or call another block of code, while saving the location of the next instruction as a point to return to.

Coprocessor instructions, which may include an instruction to load/store data to and from a coprocessor, or exchanging with CPU registers, or perform coprocessor operations.

A processor of a computer of the present system may include “complex” instructions in their instruction set. A single “complex” instruction does something that may take many instructions on other computers. Such instructions are typified by instructions that take multiple steps, control multiple functional units, or otherwise appear on a larger scale than the bulk of simple instructions implemented by the given processor. Some examples of “complex” instructions include: saving many registers on the stack at once, moving large blocks of memory, complicated integer, and floating-point arithmetic (sine, cosine, square root, etc.), SIMD instructions, a single instruction performing an operation on many values in parallel, performing an atomic test-and-set instruction or other read-modify-write atomic instruction, and instructions that perform ALU operations with an operand from memory rather than a register.

An instruction may be defined according to its parts. According to more traditional architectures, an instruction includes an opcode that specifies the operation to perform, such as add contents of memory to register—and zero or more operand specifiers, which may specify registers, memory locations, or literal data. The operand specifiers may have addressing modes determining their meaning or may be in fixed fields. In very long instruction word (VLIW) architectures, which include many microcode architectures, multiple simultaneous opcodes and operands are specified in a single instruction.

Some types of instruction sets do not have an opcode field (such as Transport Triggered Architectures (TTA) or the Forth virtual machine), only operand(s). Other unusual “0-operand” instruction sets lack any operand specifier fields, such as some stack machines including NOSC.

Conditional instructions often have a predicate field—several bits that encode the specific condition to cause the operation to be performed rather than not performed. For example, a conditional branch instruction is executed, and the branch taken, if the condition is true, so that execution proceeds to a different part of the program, and not executed, and the branch not taken, if the condition is false, so that execution continues sequentially. Some instruction sets also have conditional moves, so that the move is executed, and the data stored in the target location, if the condition is true, and not executed, and the target location not modified, if the condition is false. Similarly, IBM z/Architecture has a conditional store. Some instruction sets include a predicate field in every instruction; this is called branch predication.

The instructions constituting a program are rarely specified using their internal, numeric form (machine code); they may be specified using an assembly language or, more typically, may be generated from programming languages by compilers.

The present invention will now be more fully described by reference to the following non-limiting examples.

### Example 1: Interrogation of Aptamer-Based Biosensor by Application of a Change in Potential to the Working Electrode

This example utilized an electrochemical biosensor having an aptamer capable of selective binding to vancomycin. The aptamer was labelled with methylene blue as the redox-active reporter.

The labelled aptamers were bound to the surface of a gold electrode via a thiol linkage, with methylene blue label at the distal end of the aptamer. The gold electrode functions as the working electrode in the biosensor apparatus. Upon selective binding of vancomycin, the aptamers change conformation, thereby bringing the methylene blue labels closer to the electrode surface.

The biosensor apparatus further comprised a silver/silver chloride reference electrode and a platinum counter electrode.

The working electrode and the counter electrode were immersed in solutions containing varying concentrations of vancomycin. Using a potentiostat (PalmSens BV, Houten, Netherlands) the working electrode was held at an initial potential of −0.4 V versus the silver| silver chloride reference electrode for one second. The potential of the gold electrode was then changed in a step wise manner to −0.2 V, and maintained at that potential (again using the potentiostat) for 50 milliseconds. Current through the working electrode was recorded over the 50 milliseconds at intervals of 20 microseconds. A 20-point moving average of the current was calculated for each time point to reduce possible random variation in the current measurement.

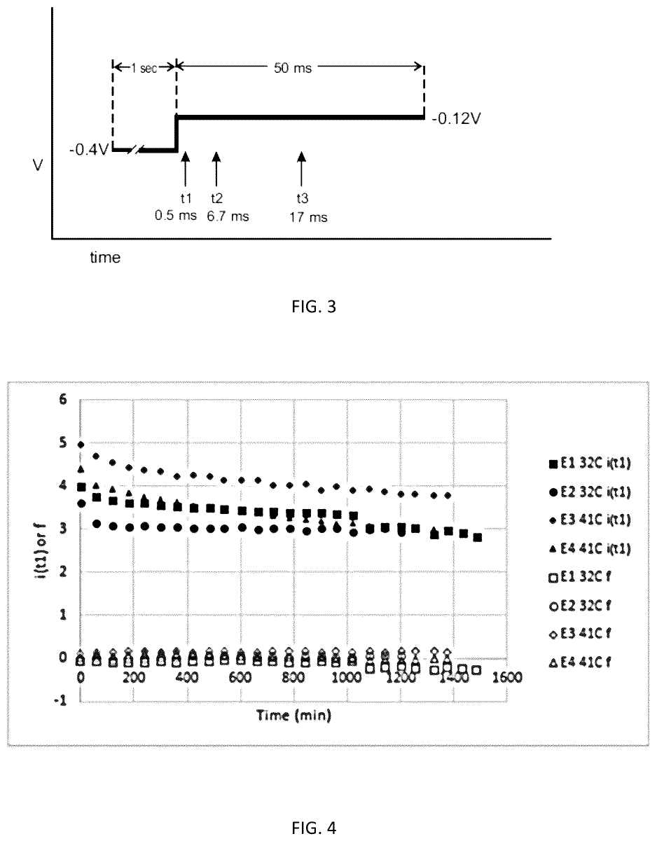

The averaged current was determined for time points at 0.5 milliseconds (i(t₁)), 6.7 milliseconds (i(t₂)), and 17 milliseconds (i(t₃)), the time points being taken by reference to the change in potential. Reference is made to FIG. 3 showing in graphical form the initial potential, the change in potential and the time points t₁, t₂ and t₃.

Current ratios were determined as follows: i(t₁)/i(t₃) and i(t₂)/i(t₃) calculated. f values were determined using Equation (3), derived using Equations (1) and (2). In Equation (3) terms up to n=10 were used to approximate the infinite series of exponentials.

$$f = - \frac{{\sum }_{n = 0}^{∞} ( \frac{2 {( - 1 )}^{n}}{( 2 n + 1 ) π} ) \cdot ( {r}_{1} {e}^{- {( 2 n + 1 )}^{2} {qt}_{3}} - {e}^{- {( 2 n + 1 )}^{2} {qt}_{1}} )}{{\sum }_{n = 0}^{∞} ( 1 - \frac{4 {( - 1 )}^{n}}{( 2 n + 1 ) π} ) \cdot ( {r}_{1} {e}^{- {( 2 n + 1 )}^{2} {qt}_{3}} - {e}^{- {( 2 n + 1 )}^{2} {qt}_{1}} )} \qquad ( 3 )$$

where r₁ is i(t₁)/i(t₃) and q is given by:

$$\frac{{π}^{2} D}{4 {l}^{2}} = \frac{ln ( {r}_{2} )}{{t}_{3} - {t}_{2}} = q .$$

It should be noted that the ratio i(t₂)/i(t₃) is present in the calculation for f, albeit not shown explicitly. To explain further, the variable r₂ is in fact:

$${r}_{2} = \frac{i ( {t}_{2} )}{i ( {t}_{3} )}$$

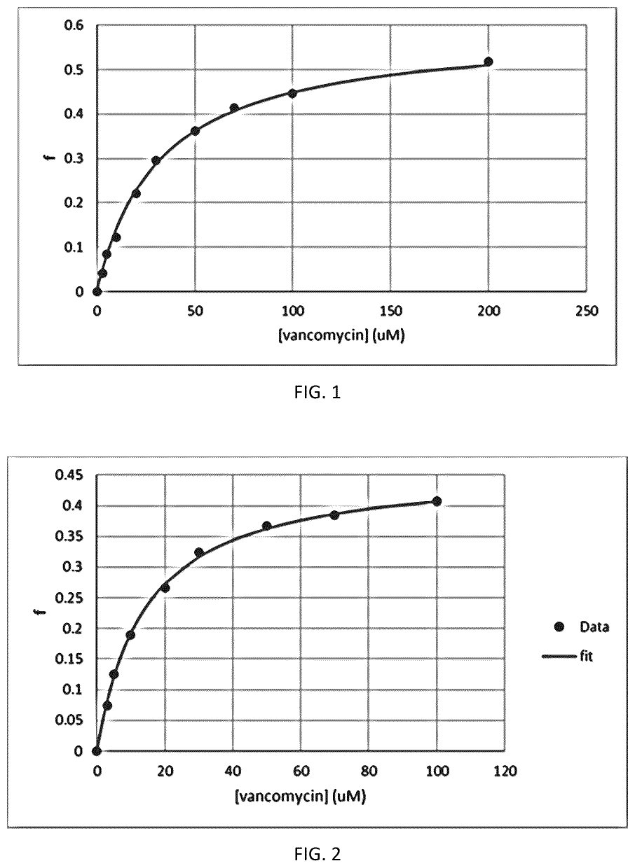

Three separate determinations of f were carried out and averaged to give the f values graphed in FIG. 1 and FIG. 2.

It was found that the calculated f values conform well to the expected Langmuir binding isotherm over the range of concentrations shown.

### Example 2: Demonstration of Low Variability in Aptamer-Based Biosensor Across a Range of Conditions and Electrodes

The experimental arrangement described in Example 1 was used in this second example to demonstrate the ability of the interrogation method to correct for electrode-to-electrode variation, temperature variation and drift in the electrode response over time.

Four different electrodes were used (E1, E2, E3 and E4). Two electrodes (E1, E2) were operated at 32° C., while the remaining electrodes (E3, E4) were operated at 41° C.

These electrodes were tested in phosphate buffered saline solutions containing no vancomycin, the target for the aptamers in the sensing layer.

Reference is made to FIG. 4 demonstrating the ability of the method to account for electrode-to-electrode variation, temperature variation and drift in the electrode response over time.

The sensor was interrogated continuously over 25 hours and the current plotted for each electrode at t₁ (0.5 ms) at approximately one-hour intervals in FIG. 4. This i(t₁) data is representative of the real variation in the sensor responses caused by electrode-to-electrode variations as manufactured, test temperature and degradation of the sensing layer on the electrode over time. The data shown is the mean of three replicate current transients.

Also shown in FIG. 4 is data from the same tests, but analysed using the present method to give f values according to Equation (3), that have been background corrected by subtracting the mean of the zero-time response across the electrodes.

These results demonstrate the ability of the invention to greatly reduce the electrode-to-electrode variation, temperature-induced variation, and signal output drift over time of the sensor response.

### Example 3: Demonstration of a Multipulse Method to Improve in Discrimination of Different Target Analyte Concentration in Aptamer-Based Biosensor

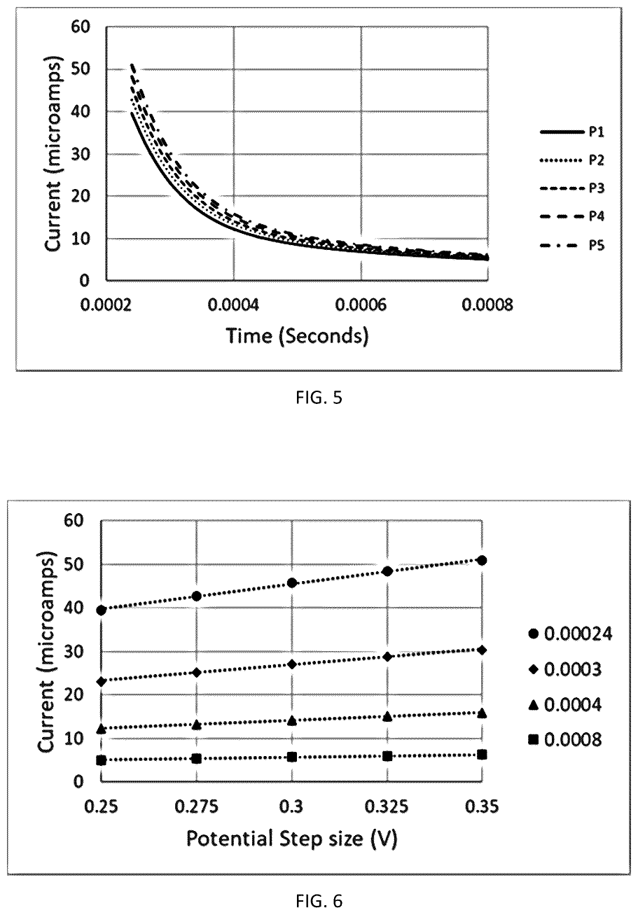

The experimental arrangement described in Example 1 was used in this third example to demonstrate the ability of a multipulse method to improve the ability to discriminate between different target analyte concentrations. The data shown in FIG. 5 to FIG. 9 are for a single sensing electrode tested in phosphate buffered saline at 37° C., in a zero to 25 micromolar solution of vancomycin. Five different interrogation pulses were used. Each of the pulses ended at a potential of −0.15 V. The first pulse started at a potential of −0.4 V, the second started at a potential of −0.425 V, the third started at a potential of −0.45 V, the fourth started at a potential of −0.475 V, and the fifth started at a potential of −0.5 V. Each of the potentials are quoted versus a silver|silver chloride reference electrode. The currents shown are the mean of five replicate pulses.

FIG. 5 shows the current recorded over time for the five different pulses, labelled P1 to P5 in the legend, when zero vancomycin is present.

Using the data shown in FIG. 5, FIG. 6 shows example plots of the current at various times (as shown in the legend in seconds) plotted against the size of the potential step used for the pulse in volts (V). The dotted lines are linear least squares regression lines. This figure demonstrates the excellent linearity of these plots across time, confirming that in this case a linear least squares regression is an appropriate technique for extrapolating the current to zero potential step size.

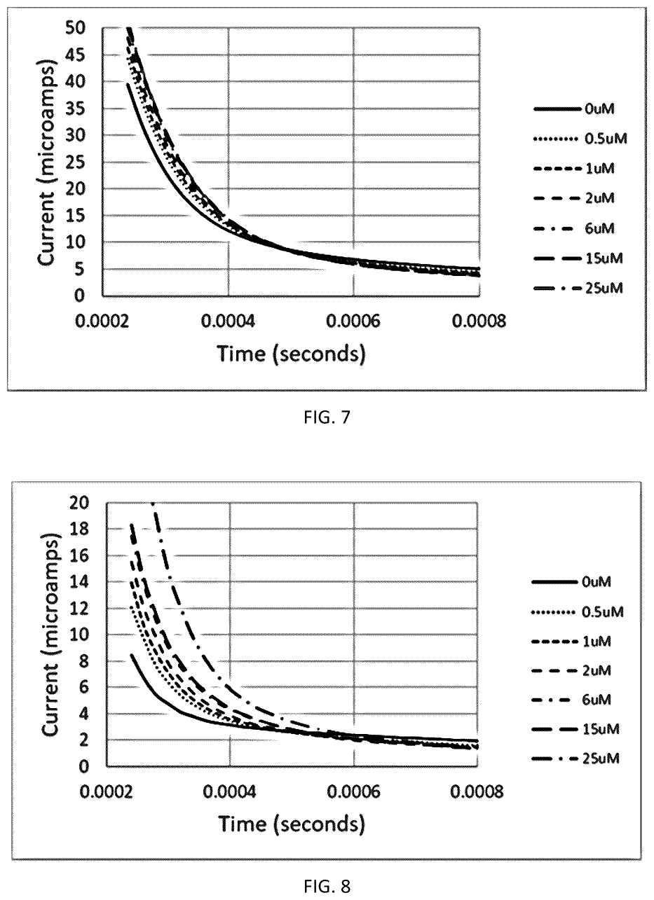

FIG. 7 shows the current versus time plots when a potential step of −0.4 V to −0.15 V is used, for various concentrations of vancomycin from zero to 25 micromolar present in the test solution, where the legend gives the concentration of vancomycin present in micromolar.

FIG. 8 shows the current extrapolated to zero potential step size using the five different pulse sizes for the same vancomycin solutions used in FIG. 7.

The improvement in the separation between the current plots for the different vancomycin concentrations in FIG. 8 compared to FIG. 7 demonstrates the ability of the multipulse method to improve the concentration discrimination in the sensor response.

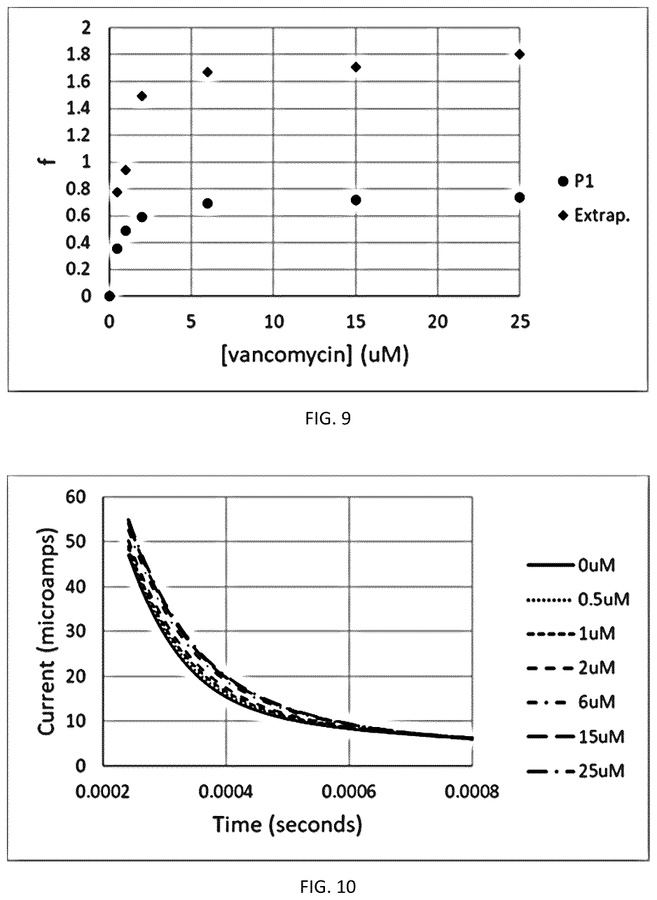

FIG. 9 shows the f values, corrected for the zero vancomycin concentration value, calculated using the currents shown in FIG. 7 (circle points labelled P1 in the legend) and those calculated using the currents shown in FIG. 8 (diamond points labelled Extrap. in the legend), demonstrating the improved dynamic range of the results and thus improved sensitivity when using the extrapolated current data.

### Example 4: Demonstration of a Dual Pulse Method to Improve in Discrimination of Different Target Analyte Concentration in Aptamer-Based Biosensor

The experimental arrangement described in Example 1 was used in this example to demonstrate the ability of a dual pulse method to improve the ability to discriminate between different target analyte concentrations. The data shown in FIG. 10 to FIG. 13 are for a single sensing electrode tested in phosphate buffered saline at 37° C., in a zero to 25 micromolar solution of vancomycin. Two different interrogation potential steps were used. The first potential step started at a potential of −0.45 V and ended at a potential of −0.2 V. The second potential started at a potential of −0.2 V and ended at −0.1 V. Each the potentials are quoted versus a silver| silver chloride reference electrode.

FIG. 10 shows the current versus time plots when a potential step of −0.45 V to −0.2 V was used, for various concentrations of vancomycin from zero to 25 micromolar present in the test solution, where the legend gives the concentration of vancomycin present in micromolar. The currents shown are the mean of five replicate potential pulses. Here is demonstrated how the current varies with vancomycin concentration.

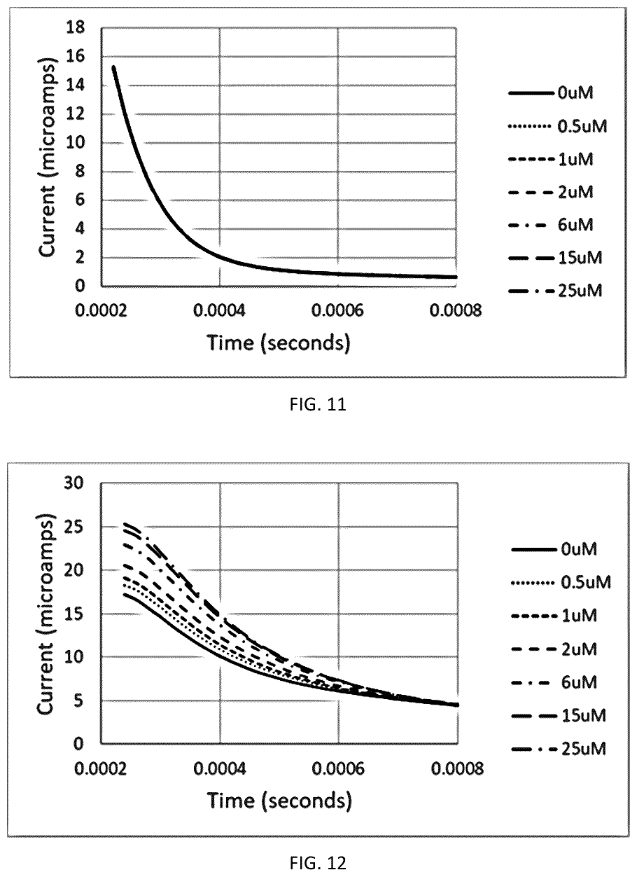

FIG. 11 shows the current versus time plots when a potential step of −0.2 V to −0.1 V was used, for various concentrations of vancomycin from zero to 25 micromolar present in the test solution, where the legend gives the concentration of vancomycin present in micromolar. The currents shown are the mean of five replicate potential pulses. Here is demonstrated how the current is substantially invariant with vancomycin concentration.

FIG. 12 shows the current versus time plots when the currents shown in FIG. 11 were multiplied by 2.5 and subtracted from the currents in FIG. 10, for various vancomycin concentrations. A factor of 2.5 was used to compensate for the potential step size used to generate the currents shown in FIG. 10 being 0.25 V, whereas a 0.1 V potential step size was used to generate the currents in FIG. 11 and where the magnitude of the capacitive charging current is expected to be proportional to the potential step size.

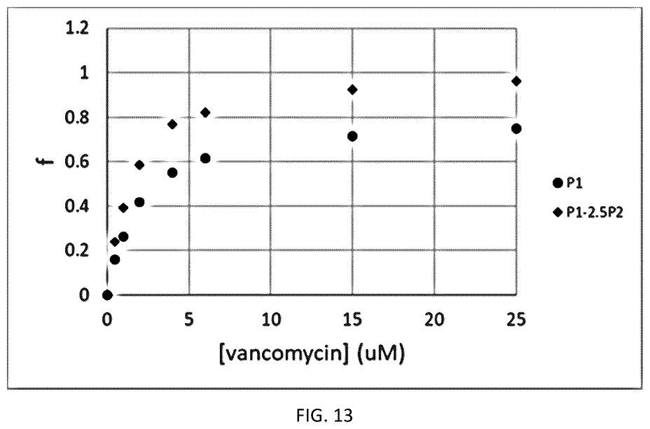

FIG. 13 shows the f values, calculated using the currents shown in FIG. 10 (circle points labelled P1 in the legend) and those calculated using the currents shown in FIG. 11 (diamond points labelled P1-2.5P2 in the legend), demonstrating the improved dynamic range of the results and thus improved sensitivity when using currents where the second potential step current was subtracted from the first potential step current. The f values have been corrected for the background value.

Those skilled in the art will appreciate that the invention described herein is susceptible to further variations and modifications other than those specifically described. It is understood that the invention comprises all such variations and modifications which fall within the spirit and scope of the present invention.

The present invention has been described mainly by reference to an aptamer-based biosensor whereby the redox reporter is covalently bound to the aptamer. The aptamer may be a DNA or RNA aptamer, or some analogue such as XNA (xeno nucleic acid) or PNA (peptide nucleic acid). Given the benefit of the present disclosure, the skilled person is enabled to practice the invention in a range of alternative formats.

For example, the binding element may be an antibody, antibody fragment (such as a Fab fragment), a natural or synthetic polymer comprising an analyte binding site, a peptide, a small molecule, an antigen, or any other element that can be bound to the electrode and specifically bind to the analyte of interest.

Moreover, the redox-active reporter need not be covalently linked to the binding element. In some embodiments, the redox-active species is the analyte itself which binds to the binding element thereby becoming tethered and spatially restricted. In other embodiments the redox-active reporter is directly bound to the analyte itself, or is associated with a separate species that is bound to the analyte, such as a redox labelled antibody.

1. A method for determining an amount of an analyte in a fluid, the method comprising:
applying a first change in potential to an electrochemical sensor working electrode, the electrode having associated therewith (i) a binding element and (ii) an associated redox-active species spatially constrained within a layer adjacent to the electrode surface;
measuring current values resulting from at least the application of the first change in potential at a plurality of time points;
determining a distribution of the redox-active species by reference to the measured current values; and
determining the amount of an analyte bound to the binding element using the determined distribution of the redox-active species.

2. The method of claim 1, wherein the change in potential is substantially instantaneous and/or effected as a step-wise change.

3. (canceled)

4. The method of claim 1, wherein the first change in potential is configured to change the redox state of the redox-active species from one redox state to another redox state such that electrons are transferred between the redox-active species and the electrode surface.

5. The method of claim 1, wherein the first change in potential is configured such that the current flowing through the electrode surface as a result of the redox-active species changing redox state is controlled by mass transport of the redox-active species through the layer adjacent to the electrode surface.

6. (canceled)

7. The method of claim 1 comprising applying an initial potential prior to the application of the first change in potential.

8. (canceled)

9. (canceled)

10. The method of claim 1 comprising measuring at least a first current value subsequent to the application of the first change in potential.

11. The method of claim 10, wherein the first current value is measured substantially immediately subsequent to the application of the first change in potential.

12. (canceled)

13. The method of claim 10, comprising measuring at least a first and a second current value in chronological order subsequent to the application of the first change in potential.

14. The method of claim 10, comprising measuring one or more of a first, a second, a third and a fourth current value in chronological order subsequent to the application of the first change in potential.

15. (canceled)

16. The method of claim 10, wherein measurement of the first current value, the second current value, the third current value and the fourth current value occurs within the period that the potential resulting from the first change in potential is maintained.

17. The method of claim 10, wherein the first current value, a second current value, a third current value and a fourth current value are averaged, the averaged current value being determined by reference to a plurality of current values determined up to, across, or after a time point.

18. (canceled)

19. The method of claim 10 comprising generating at least one current ratio by reference to the first current value and a second current value.

20. The method of claim 19, wherein the first current value is measured substantially immediately after application of the first change in potential.

21. (canceled)

22. (canceled)

23. (canceled)

24. The method of claim 1, comprising applying a second change in potential to the electrochemical sensor working electrode after the first change in potential.

25. (canceled)

26. The method of claim 24, wherein the second change in potential is used to estimate the capacitive charging current as well as any faradaic current from redox species that are not confined in the layer adjacent to the electrode, and therefore background current draw, of the sensor apparatus.

27. (canceled)

28. The method of claim 1, wherein a change in potential is a substantially instantaneous change and/or is a substantially step-wise change.

29. (canceled)

30. The method of claim 1, comprising:
applying a first change in potential and at least a second change in potential;
combining selected currents resulting from the at least second change in potential with selected currents from the first change in potential to derive a current; and
determining an analyte concentration from the derived current.

31. The method of claim 30, wherein the at least second change in potential changes from the potential applied at the end of the first change in potential.

32. (canceled)

33. (canceled)

34. The method of claim 30, comprising combining the currents resulting from the first change in potential and the at least second change in potential by using the currents measured at the same time after the change in potential and the size of the change in potential used to generate the currents, to extrapolate the measured currents to a value corresponding to a zero change in potential.

35. (canceled)

36. (canceled)

37. An electrochemical sensor apparatus or system comprising:
a working electrode having associated therewith (i) a binding element and (ii) an associated redox-active species spatially constrained within a layer adjacent to the electrode surface; and
a microprocessor-based controller,
wherein the microprocessor-based controller is configured to perform or contribute to a method for determining an amount of an analyte in a fluid about the working electrode, the method comprising:
applying a first change in potential to the working electrode, the electrode having associated therewith (i) a binding element and (ii) an associated redox-active species spatially constrained within a layer adjacent to the electrode surface;
measuring current values resulting from at least the application of the first change in potential at a plurality of time points;
determining a distribution of the redox-active species by reference to the measured current values; and
determining the amount of an analyte bound to the binding element using the determined distribution of the redox-active species.

38. (canceled)

39. (canceled)

40. (canceled)

41. (canceled)

42. (canceled)

43. (canceled)

44. (canceled)
